(19) United States
(12) Patent Application Publication
(10) Pub. No.: US 2025/0200784 A1
(43) Pub. Date: Jun. 19, 2025

(54) COMPUTING MOMENTS OF INERTIA OF OBJECTS USING FLUOROSCOPIC PROJECTION IMAGES

(71) Applicant: AURIS HEALTH, INC., Santa Clara, CA (US)

(72) Inventor: Mohammad Matinfar, San Jose, CA (US)

(73) Assignee: AURIS HEALTH, INC., Santa Clara, CA (US)

(21) Appl. No.: 18/966,094

(22) Filed: Dec. 2, 2024

Related U.S. Application Data
(60) Provisional application No. 63/609,452, filed on Dec. 13, 2023.

(51) Int. Cl. G06T 7/70; A61B 6/00; A61B 6/12; G06T 7/11; G06T 11/00

(52) U.S. Cl. CPC G06T 7/70; A61B 6/12; A61B 6/487; G06T 7/11; G06T 11/005; G06T 2207/10068; G06T 2207/10121

(57) ABSTRACT

A system includes an imaging device configured to rotate axially about a rotational axis and to capture a plurality of images associated with a sinogram at a plurality of angles, respectively, along the rotational axis, one or more processors, and a memory storing instructions that, when executed by the one or more processors, cause the system to: detect an object in each image of the plurality of images, determine a moment of inertia associated with the object based on the plurality of images, simulate one or more images for the sinogram based at least in part on the moment of inertia, generate a reconstruction of the object based on the sinogram; and determine one or more properties of the object based on the reconstruction.

### CROSS-REFERENCE TO RELATED APPLICATIONS

This application claims priority and benefit under 35 U.S.C. § 119 (e) to U.S. Provisional Patent Application No. 63/609,452, filed Dec. 13, 2023, which is incorporated herein by reference in its entirety.

### BACKGROUND

The present disclosure relates to object identification and object semantic segmentation systems. The systems can generate a complete sinogram from a plurality of fluoroscopic images containing objects projected with selected angles and, from the complete sinogram, reconstruct cross-sections of the objects. The generation of the complete sinogram may involve determining moments of inertia and principal axes from the plurality of fluoroscopic images and simulating one or more fluoroscopic images projected at other unselected angles using them. When cross-sections of the objects are reconstructed, various geometrical properties may be derived from the reconstructed cross-sections. The geometrical properties can serve to automatically segment the objects in a fluoroscopic image and, in particular, provide initial pose estimation of the objects in the fluoroscopic image.

### BACKGROUND

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a system including an imaging device, one or more processors, and a memory. The imaging device is configured to rotate axially about a rotational axis and to capture a plurality of images associated with a sinogram at a plurality of angles, respectively, along the rotational axis. The memory stores instructions that, when executed by the one or more processors, cause the system to detect an object in each image of the plurality of images, determine a moment of inertia associated with the object based on the plurality of images, simulate one or more images for the sinogram based at least in part on the moment of inertia associated with the object, generate a reconstruction of the object based on the sinogram, and determine one or more properties of the object based on the reconstruction.

Another innovative aspect of the subject matter of this disclosure can be implemented in a computer-implemented method. The method includes steps of receiving a plurality of images associated with a sinogram, where the plurality of images is captured by an imaging device at a plurality of angles, respectively, along a rotational axis of the imaging device; detecting an object in each image of the plurality of images; determining a moment of inertia associated with the object based on the plurality of images; simulating one or more images for the sinogram based at least in part on the moment of inertia associated with the object; generating a reconstruction of the object based on the sinogram; and determining one or more properties of the object based on the reconstruction.

Another innovative aspect of the subject matter of this disclosure can be implemented in a controller for a medical system, including one or more processors and a memory. The memory stores instructions that, when executed by the one or more processors, cause the controller to receive a plurality of images associated with a sinogram, where the plurality of images is captured by an imaging device at a plurality of angles, respectively, along a rotational axis of the imaging device; detect an object in each image of the plurality of images; determine a moment of inertia associated with the object based on the plurality of images; simulate one or more images for the sinogram based at least in part on the moment of inertia associated with the object; generate a reconstruction of the object based on the sinogram; and determine one or more properties of the object based on the reconstruction.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an embodiment of a robotic medical system arranged for diagnostic and/or therapeutic ureteroscopy in accordance with one or more embodiments.

FIG. 2 illustrates a robotic system arranged for diagnostic and/or therapeutic bronchoscopy in accordance with one or more embodiments.

FIG. 3 illustrates a table-based robotic system in accordance with one or more embodiments.

FIG. 4 illustrates medical system components that may be implemented in any of the medical systems of FIGS. 1-3 in accordance with one or more embodiments.

FIG. 5 illustrates an axially rotatable fluoroscopic system configured to project images of object(s) in accordance with one or more embodiments.

FIGS. 6-1, 6-2, 6-3, and 6-4 illustrate axial rotations of a fluoroscopic imaging device and corresponding captured images in accordance with one or more embodiments.

FIGS. 7-1, 7-2, and 7-3 illustrate traditional method of generating a sinogram by varying projection angles in accordance with one or more embodiments.

FIG. 8 illustrates a process for simulating a complete sinogram using select few fluoroscopic images in accordance with one or more embodiments.

FIGS. 9-1 and 9-2 illustrate an example simulated sinogram and a reconstructed cross-section of an object from the sinogram in accordance with one or more embodiments.

FIG. 10 illustrates a presentation of derived geometrical properties of a fluoroscopic image in accordance with one or more embodiments.

FIG. 11 illustrates a flowchart of a process of reconstructing an object from fluoroscopic images in accordance with one or more embodiments.

FIG. 12 shows a block diagram of an example controller for a medical system, according to some implementations.

FIG. 13 shows an illustrative flowchart depicting an example operation for determining one or more properties of an object, according to some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts features associated with separate figures that are identified by common reference numbers are not related and/or similar with respect to at least certain aspects.

The present disclosure provides systems, devices, and methods for a mathematical reconstruction of objects in a projection image, which can be used to derive various geometrical properties of the objects within the image and can serve as an initialization for object semantic segmentation. In particular, systems, devices, and methods in accordance with one or more aspects of the present disclosure can reduce a number of projection images needed to generate a complete sinogram. Additionally, the one or more aspects can serve to identify the objects in projection images and to estimate their initial poses. It is contemplated that the objects can include, but not limited to, a scope and a nodule.

With respect to medical instruments described in the present disclosure, the term “instrument” is used according to its broad and ordinary meaning and may refer to any type of tool, device, assembly, system, subsystem, apparatus, component, or the like. In some contexts herein, the term “device” may be used substantially interchangeably with the term “instrument.”

Robotic surgical systems can be utilized to facilitate instrument navigation to areas within a patient's body. In some embodiments, robotic systems can be configured to provide an interface that allows an operator to navigate robotically-controlled instrumentation by directing the movement of the instrumentation in multiple degrees of freedom. For example, the operator may direct axial translation (i.e., insertion and/or retraction), articulation angle, and/or roll (i.e., articulation angle direction), of endoscopes, access sheaths, guidewires, working instruments (e.g., needles, baskets, lithotripsy devices, etc.). Navigation within organs, branch vessel ostia, or other relatively open three-dimensional space can be challenging due to the need to understand the three-dimensional relationship of the navigated/tracked instrument relative to the anatomical target and/or to determine in which plane the instrument will bend. This task can be difficult, in part, because navigating an instrument through a lumen of the patient from a remote patient access point to the desired site of a procedure requires manipulating the instrument without a direct line of sight of the instrument. A positioning/tracking system may be used to help locate the desired site of the procedure and visualize the navigation of the instrument to the desired site of the procedure. Positioning/tracking systems allow the user to visualize a patient's internal anatomy and the location and/or orientation of the detectable markers of the instrument within the patient's anatomy.

Positioning systems can include imaging systems/modalities, such as positron emission tomography (PET), X-ray computed tomography (CT), X-ray fluoroscopy, magnetic resonance imaging (MRI), camera-based optical systems, and ultrasonic or other sonic imaging systems. Positioning system can further include electromagnetic (EM) tracking systems (e.g., using electromagnetic field generators as described in detail herein), fiber optic tracking systems, and robotic tracking/positioning based on robotic data (e.g., robotic actuator, torque, pose data). Some imaging systems/modalities are not suitable for continuous real-time tracking of instruments, such as PET, CT, and MRI, which generally produce and combine many cross-sectional images of an object to generate a computer-processed image; such an image capture process can be relatively slow, and movement within the image field during the image capture process can produce image artifacts that make such systems unsuitable for real-time tracking of moving instruments in a body. Additionally, some imaging systems/modalities such as X-ray, CT, and fluoroscopy emit potentially harmful ionizing radiation, and it may be desirable to limit the duration of their use.

Electromagnetic (EM) tracking systems and fiber optic tracking systems can provide real-time instrument tracking. EM tracking generally functions by detecting/determining the position/orientation of EM sensing coil(s) (i.e., an EM marker/sensor) in a fluctuating magnetic field. The fluctuating magnetic field induces a current in the coil based on the coil's position and orientation within the field. The coil's position and orientation can thus be determined by measuring the current in the coil. In some cases, a single EM sensor/marker is able to sense its position and orientation in three-dimensional space with five degrees of freedom. That is, the EM sensor can provide data indicating orientation in every direction except around the axial symmetric axis of the coil (i.e., roll). Two EM sensors/markers held in a fixed relative position and orientation on an instrument or other marker device may be used to sense all six degrees of freedom of the instrument. In navigation systems employing EM tracking, an image of an anatomical space can be acquired, wherein the system control circuitry is configured to determine a registration between a frame of reference of the EM sensor(s)/marker(s) associated with a tracked instrument and a frame of reference of an imaging system/modality used to image the anatomical space to depict movement of the tracked instrument within the imaged anatomical space.

Although certain aspects of the present disclosure are described in detail herein in the context of bronchoscopy and ureteroscopy procedures, it should be understood that such context is provided for convenience and clarity, and instrument positioning concepts disclosed herein are applicable to any suitable medical procedures.

With respect to ureteroscopy procedures, surgeons may insert an endoscope (e.g., ureteroscope) into the urinary tract through the urethra to remove urinary stones from the bladder and ureter, such as for the purpose of removing kidney stones. In some procedures, physicians may use a percutaneous nephrolithotomy (“PCNL”) technique that involves inserting a nephroscope through the skin (i.e., percutaneously) and intervening tissue to provide access to the treatment site for breaking-up and/or removing the stone(s). Relatively large kidney stones can be broken into a relatively smaller fragments to facilitate extraction thereof using certain instrumentation, such as laser lithotripsy devices. According to some procedures, a basketing device/system may be used to capture the relatively smaller stone fragment(s) and extract them from the treatment site out of the patient. Any of the instrumentation associated with such ureteroscopy procedures can be robotically-controlled and/or positionally tracked by tracking/detecting marker(s)/sensor(s) associated with the instrumentation using a positioning modality as described in detail herein.

### Medical System

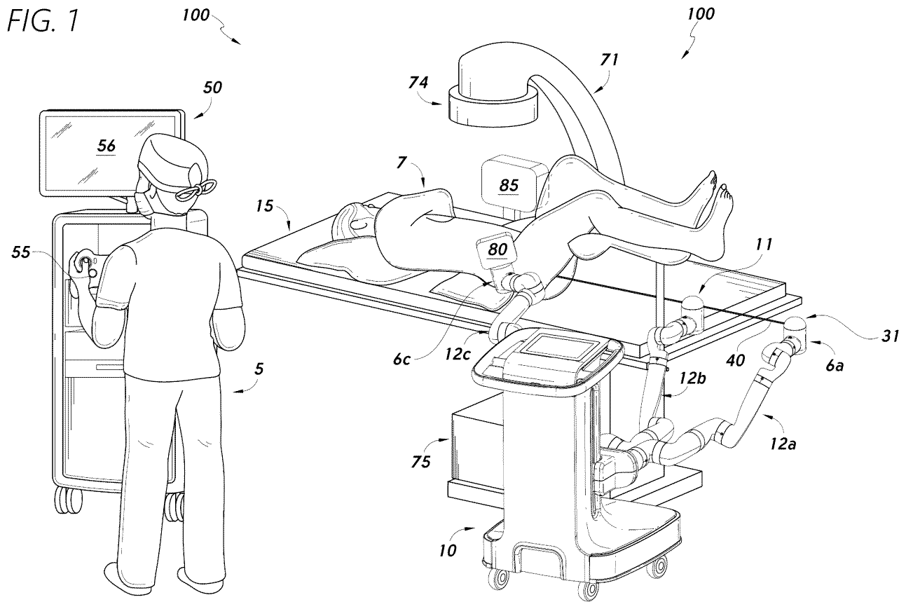

FIG. 1 illustrates an example medical system 100 for performing various medical procedures in accordance with aspects of the present disclosure. The medical system 100 may be used for, for example, endoscopic procedures. Robotic medical solutions can provide relatively higher precision, superior control, and/or superior hand-eye coordination with respect to certain instruments compared to strictly-manual procedures. Although the system 100 of FIG. 1 is presented in the context of a ureteroscopic procedure, it should be understood that the principles disclosed herein may be implemented in any type of endoscopic procedure.

The medical system 100 includes a robotic system 10 (e.g., mobile robotic cart) configured to engage with and/or control a medical instrument (e.g., ureteroscope) including a proximal handle 31 and a shaft 40 coupled to the handle 31 at a proximal portion thereof to perform a procedure on a patient 7. It should be understood that the instrument 40 may be any type of shaft-based medical instrument, including an endoscope (such as a ureteroscope or bronchoscope), catheter (such as a steerable or non-steerable catheter), needle, nephroscope, laparoscope, or other type of medical instrument. The instrument 40 may access the internal patient anatomy through direct access (e.g., through a natural orifice) and/or through percutaneous access via skin/tissue puncture.

The medical system 100 includes a control system 50 configured to interface with the robotic system 10, provide information regarding the procedure, and/or perform a variety of other operations. For example, the control system 50 can include one or more display(s) 56 configured to present certain information to assist the physician 5 and/or other technician(s) or individual(s). The medical system 100 can include a table 15 configured to hold the patient 7. The system 100 may further include an electromagnetic (EM) field generator, such as a robot-mounted EM field generator 80 or an EM field generator 85 mounted to the table 15 or other structure.

Although the various robotic arms 12 are shown in various positions and coupled to various tools/devices, it should be understood that such configurations are shown for convenience and illustration purposes, and that the medical system 100 may include any number of robotic arms 12 which may have different configurations over time and/or at different points during a medical procedure. Furthermore, the robotic arms 12 may be coupled to different devices/instruments than shown in FIG. 1, and in some cases or periods of time, one or more of the arms may not be utilized or coupled to a medical instrument. Instrument coupling to the robotic system 10 may be via robotic end effectors 6a-6c associated with distal ends of the respective arms 12. The term “end effector” is used herein according to its broad and ordinary meaning and may refer to any type of robotic manipulator device, component, and/or assembly. The terms “robotic manipulator” and “robotic manipulator assembly” are used according to their broad and ordinary meanings, and may refer to a robotic end effector and/or sterile adapter or other adapter component coupled to the end effector, either collectively or individually. For example, “robotic manipulator” or “robotic manipulator assembly” may refer to an instrument device manipulator (IDM) including one or more drive outputs, whether embodied in a robotic end effector, adapter, and/or other component(s).

In some embodiments, the physician 5 can interact with the control system 50 and/or the robotic system 10 to cause/control the robotic system 10 to advance and navigate the medical instrument shaft 40 (e.g., a scope) through the patient anatomy to the target site and/or perform certain operations using the relevant instrumentation. The control system 50 can provide information via the display(s) 56 that is associated with the medical instrument 40, such as real-time endoscopic images captured therewith, and/or other instruments of the system 100, to assist the physician 5 in navigating/controlling such instrumentation. The control system 50 may provide imaging/positional information to the physician 5 that is based on certain positioning modalities, such as fluoroscopy, ultrasound, optical/camera imaging, EM field positioning, or other modality, as described in detail herein.

The various scope/shaft-type instruments disclosed herein, such as the shaft 40 of the system 100, can be configured to navigate within the human anatomy, such as within a natural orifice or lumen of the human anatomy. The terms “scope” and “endoscope” are used herein according to their broad and ordinary meanings, and may refer to any type of elongate (e.g., shaft-type) medical instrument having image generating, viewing, and/or capturing functionality and being configured to be introduced into any type of organ, cavity, lumen, chamber, or space of a body. A scope can include, for example, a ureteroscope (e.g., for accessing the urinary tract), a laparoscope, a nephroscope (e.g., for accessing the kidneys), a bronchoscope (e.g., for accessing an airway, such as the bronchus), a colonoscope (e.g., for accessing the colon), an arthroscope (e.g., for accessing a joint), a cystoscope (e.g., for accessing the bladder), colonoscope (e.g., for accessing the colon and/or rectum), borescope, and so on. Scopes/endoscopes, in some instances, may comprise an at least partially rigid and/or flexible tube, and may be dimensioned to be passed within an outer sheath, catheter, introducer, or other lumen-type device, or may be used without such devices. Endoscopes and other instruments described herein can have associated with distal ends or other portions thereof certain markers/sensors configured to be visible/detectable in a field/space associated with one or more positioning (e.g., imaging) systems/modalities.

The system 100 is illustrated as including a fluoroscopy system 70, which includes an X-ray generator 75 and an image detector 74 (referred to as an “image intensifier” in some contexts; either component 74, 75 may be referred to as a “source” or “emitter” herein), which may both be mounted on a moveable C-arm 71. In some instances, the fluoroscopy system 70 and any portions thereof may be referred as an imaging device. The control system 50 or other system/device may be used to store and/or manipulate images generated using the fluoroscopy system 70. In some embodiments, the bed 15 is radiolucent, such that radiation from the generator 75 may pass through the bed 15 and the target area of the patient's anatomy, wherein the patient 7 is positioned between the ends of the C-arm 71. The structure/arm 71 of the fluoroscopy system 70 may be rotatable or fixed. The fluoroscopy system 70 may be implemented to allow live images to be viewed to facilitate image-guided surgery. The structure/arm 71 can be selectively moveable to permit various images of the patient 7 and/or surgical field to be taken by the fluoroscopy panel source 74.

In the example urology configuration shown in FIG. 1, the robotic arm 12c is shown holding the field generator 80. As the electric field generated by the electric field generator 80 can be distorted by the presence of metal or other conductive components therein, it may be desirable to position the arm 12c in a manner such that other components of the system do not interfere substantially with the electric field. For example, it may be desirable to position the field generator 80 at least 8″ or more away from the support arm 71 associated with the fluoroscopy system. In some embodiments, the system 100 includes an EM field generator 85 mounted to the table 15 or other structure (e.g., stand-alone structure).

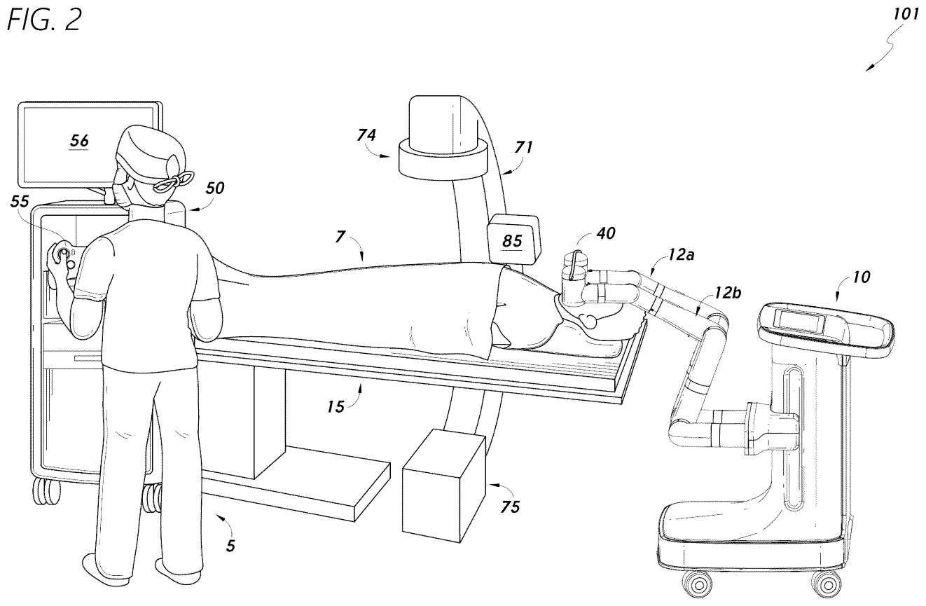

FIG. 2 illustrates a cart-based robotic system 101 arranged for diagnostic and/or therapeutic bronchoscopy in accordance with one or more embodiments. During a bronchoscopy, the arm(s) 12 of the robotic system 10 may be configured to drive a medical instrument shaft 40, such as a steerable endoscope, which may be a procedure-specific bronchoscope for bronchoscopy, through a natural orifice access point (e.g., the mouth of the patient 7 positioned on a table 15 in the present example) to deliver diagnostic and/or therapeutic tools. As shown, the robotic system 10 (e.g., cart) may be positioned proximate to the patient's upper torso in order to provide access to the access point. The arrangement in FIG. 2 may also be utilized when performing a gastro-intestinal (GI) procedure with a gastroscope.

As with the system 100 of FIG. 1, the instrument/scope 40 may access the target anatomy through an access sheath. For surgical bronchoscopy, the endoscope 40 may be directed down the patient's trachea and lungs after insertion using precise commands from the robotic system 10 until reaching the target operative site. For example, the endoscope 40 may be directed to deliver a biopsy needle to a target, such as, for example, a lesion or nodule within the lungs of a patient.

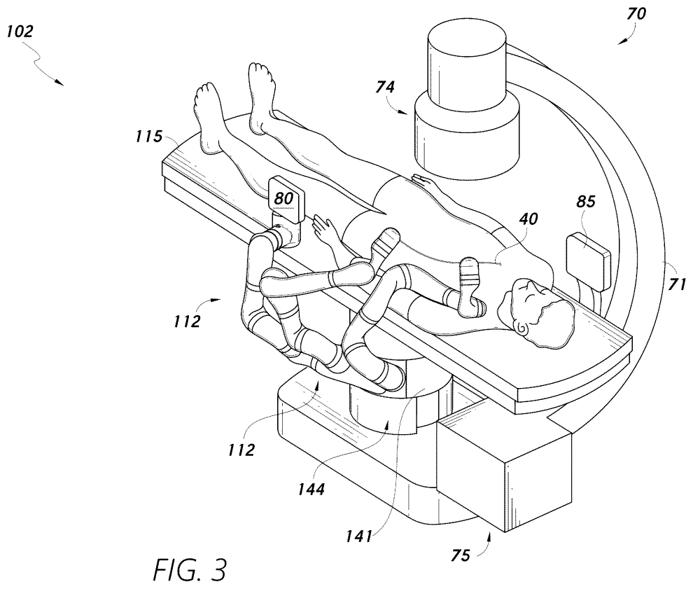

FIG. 3 illustrates a table-based robotic system 102 in accordance with one or more embodiments of the present disclosure. The system 102 incorporates robotic components integrated with a table/platform 115, thereby allowing for a reduced amount of capital equipment within the operating room compared to some cart-based robotic systems. Table-integrated robotic systems like the system 102 can allow greater access to patients in some instances. Much like in the cart-based systems 101, 102, the instrument device manipulator assemblies associated with the robotic arms 112 of the system 102 may generally comprise instruments and/or instrument feeders that are designed to manipulate an elongated medical instrument/shaft, such as a catheter 40 or the like.

As shown, the robotic-enabled table system 104 can include a column 144 coupled to one or more carriages 141 (e.g., ring-shaped movable structures), from which the one or more robotic arms 112 may emanate. The carriage(s) 141 may translate along a vertical column interface that runs at least a portion of the length of the column 144 to provide different vantage points from which the robotic arms 112 may be positioned. The carriage(s) 141 may rotate around the column 144 in some embodiments to allow the robotic arms 112 to have access to multiples sides of the table 104. Rotation and/or translation of the carriage(s) 141 can allow the system 102 to align the medical instruments, such as endoscopes and catheters, into different access points on the patient.

With reference to FIGS. 1-3 and FIG. 4, which shows example embodiments components of subsystems of any of FIGS. 1-3, the control system 50 can be configured to provide various functionality to assist in performing a medical procedure. The control system 50 can communicate with the robotic system 10 via a wireless or wired connection (e.g., to control the robotic system 10). In some embodiments, the control system 50 can communicate with the robotic system 10 to receive position data therefrom relating to the position of the distal end of the scope 40 or other instrumentation. Such positioning data may be derived using one or more markers (e.g., electromagnetic sensors, radiopaque markers, etc.) associated with the respective instrumentation and/or based at least in part on robotic system data (e.g., arm position/pose data, known parameters or dimensions of the various system components, etc.). In some embodiments, the control system 50 can communicate with the EM field generator 80/85 to control generation of an EM field in an area around the patient 7 and/or around the tracked instrumentation.

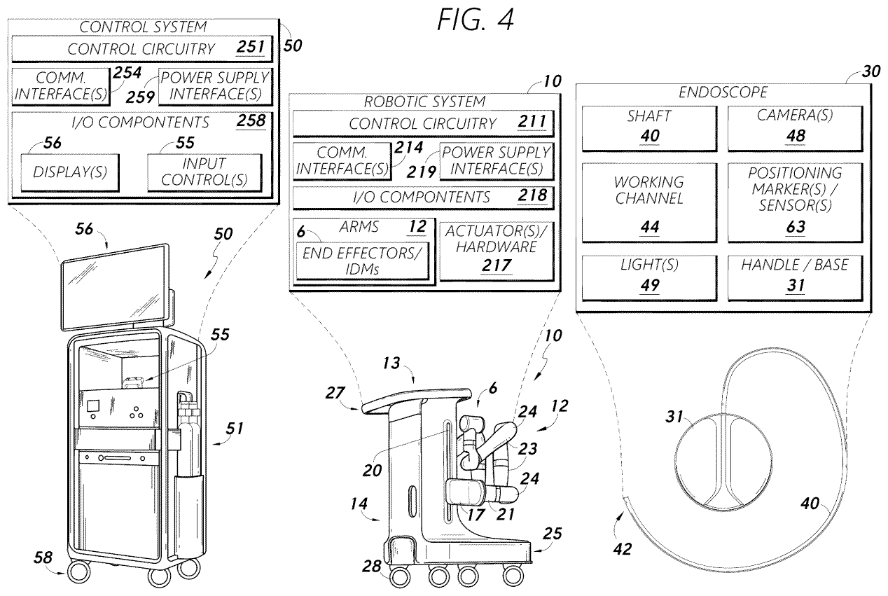

FIG. 4 further shows an example embodiment of the robotic systems 10 of any of FIGS. 1-3. The robotic system 10 can include one or more robotic arms 12, each of which can include multiple arm segments 23 coupled to joints 24, which can provide multiple degrees of movement/freedom. When the robotic system 10 is properly positioned, the scope 40 can be inserted into the patient 7 robotically using the robotic arms 12, manually by the physician 5, or a combination thereof. One of the arms 112 may have associated therewith an instrument coupling/manipulator 31 that is configured to facilitate advancement and operation of the scope 40.

The robotic system 10 can be physically and/or communicatively coupled to any component of the medical system, such as to the control system 50, the table 15, the EM field generator 80/85, the scope 40, the fluoroscopy system 70, and/or any type of percutaneous-access instrument (e.g., needle, catheter, nephroscope, etc.). For example, the robotic system 10 can include communication interface(s) 214 for communicating with communication interface(s) 254 of the control system 50. The robotic system 10 may be configured to receive control signals from the control system 50 to perform certain operations, such as to position one or more of the robotic arms 12, manipulate the scope 40, and so on. In response, the robotic system 10 can control, using certain control circuitry 211, actuators 217, and/or other components of the robotic system 10 to perform the operations. For example, the control circuitry 211 may control various motors/actuators associated with the various joints of the robotic arms 12 and/or the arm support 17. In some embodiments, the robotic system 10 and/or control system 50 is/are configured to receive images and/or image data from the scope 40 representing internal anatomy of the patient 7 and/or portions of the access sheath or other device components.

The robotic system 10 generally includes an elongated support structure 14 (also referred to as a “column”), a robotic system base 25, and a console 13 at the top of the column 14. The column 14 may include one or more arm supports 17 (also referred to as a “carriage”) for supporting the deployment of the one or more robotic arms 12 (three shown in FIG. 1). The arm support 17 may be configured to vertically translate along the column 14. In some embodiments, the arm support 17 can be connected to the column 14 through slots 20 that are positioned on opposite sides of the column 14 to guide the vertical translation of the arm support 17. The slot 20 contains a vertical translation interface to position and hold the arm support 17 at various vertical heights relative to the robotic system base 25. The base 25 balances the weight of the column 14, arm support 17, and arms 12 over the floor.

The robotic arms 12 may generally comprise robotic arm bases 21 and end effectors 6a-6c, separated by a series of linking arm segments 23 that are connected by a series of joints 24, each joint comprising one or more independent actuators 217. Each actuator may comprise an independently-controllable motor. Each independently-controllable joint 24 can provide or represent an independent degree of freedom available to the robotic arm. In some embodiments, each of the arms 12 has seven joints, and thus provides seven degrees of freedom, including “redundant” degrees of freedom. Redundant degrees of freedom allow the robotic arms 12 to position their respective end effectors 6a-6c at a specific position, orientation, and trajectory in space using different linkage positions and joint angles. This allows for the system to position and direct a medical instrument from a desired point in space while allowing the physician to move the arm joints into a clinically advantageous position away from the patient to create greater access, while avoiding arm collisions. Positioned at the upper end of column 14, the console 13 can provide both a user interface for receiving user input and a display screen 56 (or a dual-purpose device such as, for example, a touchscreen) to provide the physician/user with both pre-operative and intra-operative data. The robotic cart 10 can further include a handle 27, as well as one or more wheels 28.

The end effectors 6a-6c of each of the robotic arms 12 may comprise, or be configured to have coupled thereto, an instrument device manipulator (IDM; e.g., scope handle 31), which may be attached using a sterile adapter component in some instances. The combination of the end effectors 6a-6c and associated IDM, as well as any intervening mechanics or couplings (e.g., sterile adapter), can be referred to as a manipulator assembly. An IDM can provide power and control interfaces. For example, the interfaces can include connectors to transfer pneumatic pressure, electrical power, electrical signals, and/or optical signals from the robotic arm 12 to the IDM. The IDMs may be configured to manipulate medical instruments (e.g., surgical tools/instruments), such as the scope 40, using techniques including, for example, direct drives, harmonic drives, geared drives, belts and pulleys, magnetic drives, and the like. The robotic system 10 also may include one or more power supply interface(s) 219.

As referenced above, the system 100 can include certain control circuitry configured to perform certain of the functionality described herein, including the control circuitry 211 of the robotic system 10 and the control circuitry 251 of the control system 50. That is, the control circuitry of the systems 100, 101, 102 may be part of the robotic system 10, the control system 50, or some combination thereof. Therefore, any reference herein to control circuitry may refer to circuitry embodied in a robotic system, a control system, or any other component of a medical system, such as the medical systems 100, 101, and 102 shown in FIGS. 1-3, respectively. The term “control circuitry” is used herein according to its broad and ordinary meaning, and may refer to any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein may further include one or more circuit substrates (e.g., printed circuit boards), conductive traces and vias, and/or mounting pads, connectors, and/or components. Control circuitry referenced herein may further comprise one or more storage devices, which may be embodied in a single memory device, a plurality of memory devices, and/or embedded circuitry of a device. Such data storage may comprise read-only memory, random access memory, volatile memory, non-volatile memory, static memory, dynamic memory, flash memory, cache memory, data storage registers, and/or any device that stores digital information. It should be noted that in embodiments in which control circuitry comprises a hardware and/or software state machine, analog circuitry, digital circuitry, and/or logic circuitry, data storage device(s)/register(s) storing any associated operational instructions may be embedded within, or external to, the circuitry comprising the state machine, analog circuitry, digital circuitry, and/or logic circuitry.

The control circuitry 211, 251 may comprise computer-readable media storing, and/or configured to store, hard-coded and/or operational instructions corresponding to at least some of the steps and/or functions illustrated in one or more of the present figures and/or described herein. Such computer-readable media can be included in an article of manufacture in some instances. The control circuitry 211/251 may be entirely locally maintained/disposed or may be remotely located at least in part (e.g., communicatively coupled indirectly via a local area network and/or a wide area network). Any of the control circuitry 211, 251 may be configured to perform any aspect(s) of the various processes disclosed herein.

With further reference to FIG. 4, the robotic system 10 can provide one or more input/output (I/O) components 218, such as a user interface for receiving user input or a display screen (or a dual-purpose device, such as a touchscreen) to provide the physician or user with preoperative or intraoperative data. The control system 50 can also include various I/O components 258 configured to assist the physician 5 or others in performing a medical procedure. For example, the I/O components 258 can be configured to allow for user input to control/navigate the scope 40 and/or basketing system within the patient 7. In some embodiments, for example, the physician 5 can provide input to the control system 50 and/or robotic system 10, wherein in response to such input, control signals can be sent to the robotic system 10 to manipulate the scope 40 and/or other robotically-controlled instrumentation.

The control system 50 and/or robotic system 10 can include certain user controls (e.g., controls 55), which may comprise any type of user input (and/or output) devices or device interfaces, such as one or more buttons, keys, joysticks, handheld controllers (e.g., video-game-type controllers), computer mice, trackpads, trackballs, control pads, and/or sensors (e.g., motion sensors or cameras) that capture hand gestures and finger gestures, touchscreens, and/or interfaces/connectors therefore. Such user controls are communicatively and/or physically coupled to respective control circuitry. The control system can include a structural tower 51, as well as one or more wheels 58 that support the tower 51. The control system 50 can further include certain communication interface(s) 254 and/or power supply interface(s) 259.

In some embodiments, the endoscope assembly 30 includes a handle or base 31 coupled to an endoscope shaft 40 (referred to simply as an “endoscope,” or “scope” in certain contexts herein). For example, the endoscope 40 can include an elongate shaft including one or more lights 49 and one or more cameras or other imaging devices 48. The scope 40 can further include one or more working channels 44, which may run a length of the scope 40.

The scope assembly 30 can further comprise one or more positioning markers and/or sensors 63, which may be configured to generate signals indicating a position of the marker(s)/sensor(s) 63 within an electromagnetic field. Such markers 63 may comprise, for example, one or more conductive coils (or other embodiment of an antenna), which may be disposed at a known, fixed orientation relative to one another to allow for the determination of multiple degrees of freedom with respect to position determination. The marker(s) 63 can be configured to generate and/or send sensor position data to another device and/or produce a detectable distortion or signature in an electromagnetic field. The sensor/marker position data can indicate a position and/or orientation of the medical instrument 40 (e.g., the distal end 42 thereof) and/or can be used to determine/infer a position/orientation of the medical instrument.

The scope 40 can be articulable, such as with respect to at least a distal portion 42 of the scope 40, so that the scope 40 can be steered within the human anatomy. In some embodiments, the scope 40 is configured to be articulated with, for example, six degrees of freedom, including XYZ coordinate movement, as well as pitch, yaw, and roll. Certain position sensor(s) (e.g., electromagnetic sensors) of the scope 40, where implemented, may likewise have similar degrees of freedom with respect to the positional information they generate/provide.

For robotic implementations, robotic arms of a robotic system can be configured/configurable to manipulate the scope 40. For example, an instrument device manipulator (e.g., scope handle) can be coupled to an end effector of a robot arm and can manipulate the scope 40 using elongate movement members. The elongate movement members may include one or more pull wires (e.g., pull or push wires), cables, fibers, and/or flexible shafts. For example, the robotic end effector may be configured to actuate multiple pull wires (not shown) coupled to the scope 40 to deflect the tip 42 of the scope 40.

In various embodiments, the anatomical space in which the scope 40 or other instrument may be localized (i.e., where position of the scope/instrument is determined/estimated) is a three-dimensional portion of a patient's vasculature, tracheobronchial airways, urinary tract, gastrointestinal tract, or any organ or space accessed via such lumens. Various positioning/imaging modalities may be implemented to provide images/representations of the anatomical space. Suitable imaging subsystems include, for example, X-ray, fluoroscopy, CT, PET, PET-CT, CT angiography, Cone-Beam CT, 3DRA, single-photon emission computed tomography (SPECT), MRI, Optical Coherence Tomography (OCT), and ultrasound. One or both of pre-procedural and intra-procedural images may be acquired. In some embodiments, the pre-procedural and/or intra-procedural images are acquired using a C-arm fluoroscope. In connection with some embodiments, particular positioning and imaging systems/modalities are described; it should be understood that such description may relate to any type of positioning system/modality.

### Capture of Multiple Images

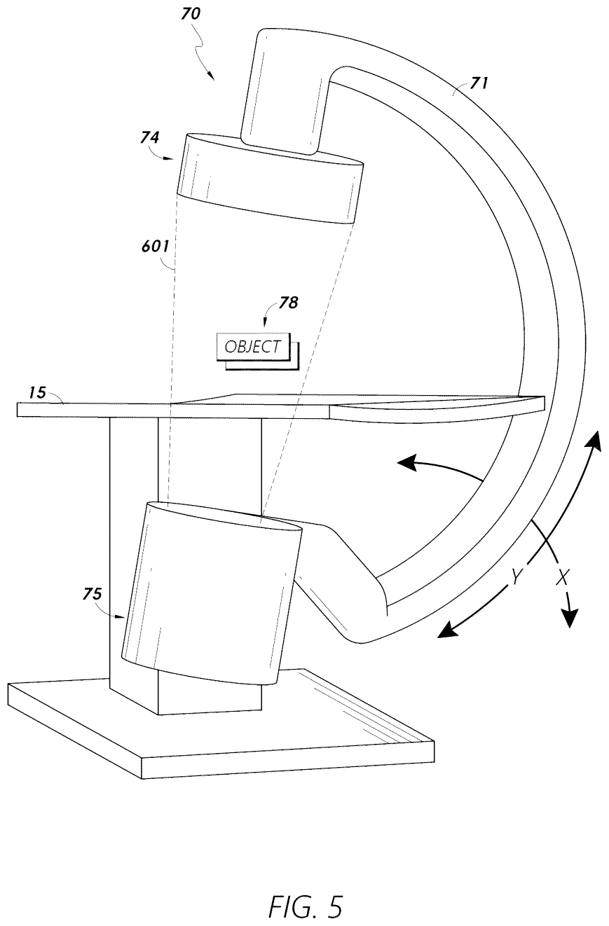

FIG. 5 illustrates an axially rotatable fluoroscopic system 70 configured to project images of object(s) in accordance with one or more embodiments. In the present disclosure, “projecting” a fluoroscopic image may be synonymously used for “capturing” a fluoroscopic image as a fluoroscopic imaging device projects a fluoroscopic field onto a region of interest to capture objects within the region of interest. The fluoroscopic system 70 can include a mounting arm, such as a C-arm 71, or other structure holding/supporting a fluoroscopic imaging device. The fluoroscopic imaging device can include a detector/receiver 74 or source/emitter 75. While the C-arm 71 is illustrated for the fluoroscopic system 70, it is to be understood that other supporting structures and imaging devices are contemplated.

In the fluoroscopy system 70, the source/emitter 75 and the detector/receiver 74 may be positioned at opposing ends of the C-arm 71 to maintain a fixed relationship in position and orientation. The source/emitter 75 can generate and the detector/receiver 74 can receive a fluoroscopy field (e.g., X-ray) 601 to record intensity of the fluoroscopic field that pass through an object 78 positioned on the bed 15. Areas of the object 78 that are denser attenuates more of the fluoroscopy field and results in lower intensity on the detector/receiver 74, and vice versa. As shown, the C-arm 71 may be axially rotated about a center of rotation along one or more axes. For example, the illustrated C-arm 71 may be rotated along the ‘X’ axis (e.g., to provide horizontally rotated images), the ‘Y’ axis (e.g., to provide vertically rotated images), or both. The object 78, which can be any semantically segmentable object, can be positioned at or near the center of rotation of the C-arm 71. Some example semantically segmentable objects include, without limitation, a scope, a medical tool (e.g., a biopsy needle, basket, forceps, etc.), and an anatomical feature.

Axially rotating the C-arm 71 to capture multiple projected images at various angles can provide a more complete two-dimensional or three-dimensional understanding of various geometrical properties of the object 78 than a single image may provide. However, capturing multiple projected images can be time and resource intensive. Accordingly, an improved technique that can efficiently and reliably derive various geometrical properties using a minimum number of projected images is desired.

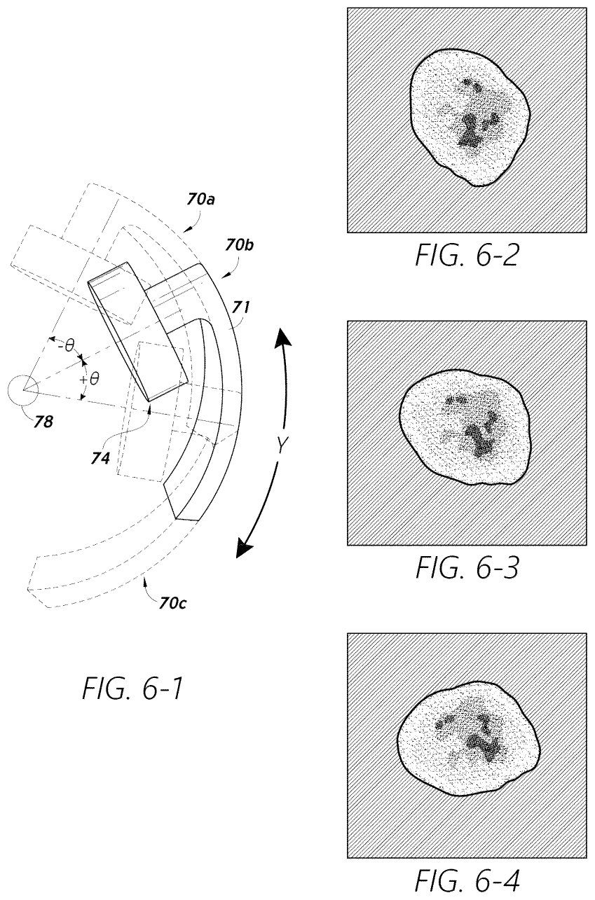

FIGS. 6-1, 6-2, 6-3, and 6-4 illustrate axial rotations of a fluoroscopic imaging device and corresponding captured images in accordance with one or more embodiments. FIG. 6-1 illustrates the fluoroscopy system 70 attached to the C-arm 71 including the emitter/source 75 or the detector/receiver 74. As shown, the device-arm 71 can be axially rotated along a ‘Y’ axis to provide a neutral configuration 70b (0°), a negative configuration 70c indicated with a negative angular offset (−θ), and a positive configuration 70a indicated with a positive angular offset (+θ). It is to be understood that an axial rotation of the imaging device can be along a different axis, such as the ‘X’ axis shown in FIG. 5, or multiple axes. The positive configuration 70a and the negative configuration 70c may be defined along any arbitrary rotational axis in relation to the neutral configuration 70b. While the rotational axis can be arbitrary, the positive angular offset +θ and the negative angular offset −θ may be selected to have the same or substantially the same size (e.g., |+θ|=|−θ|) along the rotational axis such that they are symmetrically positioned in relation to the neutral configuration 70b. In some embodiments, the neutral configuration may be synonymously referred to as “a reference configuration,” “a reference angle,” “a reference point,” “a zero angle,” or the like.

In some instances, a conventional angle frequently used for a C-arm or environment setup may be selected to serve as the neutral configuration 70b. For instance, either of 0° or 90° configurations, referred as “anterior posterior” or “lateral” configurations, may be such conventional angles used as the neutral configuration 70b. The conventional angles may be clinically preferred for their ease of identification and reference.

The angular offset θ between the positive configuration 70a and neutral configuration 70b or the negative configuration 70c and neutral configuration 70b can be any angular separation as long as they are substantially the same in size. For instance, when a positive angular offset +θ is selected to be +5°, +10°, +15°, +30°, +45°, or the like, a corresponding negative angular offset −θ may be, respectively, −5°, −10°, −15°, −30°, −45°, or the like. Those two angles are to be positioned along the same rotational axis defined by the positive angle and be symmetric in relation to the neutral configuration 70b. While any angles may be used, for computational efficiency, angles that are different enough (>15°) to show some variability in projected cross-sections but not too variable to show too little similarity (<30°) are typically preferred.

FIGS. 6-2, 6-3, and 6-4 illustrate fluoroscopic images each depicting a cross-section of a bone projected at an angle corresponding to the configurations 70a-c. From the neutral configuration 70b, the C-arm 71 may be rotated about an axis by a first angular offset −θ to arrive at the negative configuration 70a and, similarly, the C-arm 71 may be rotated about the axis by a second angular offset +° to arrive at the positive configuration 70c. Additional image(s) may be projected from the negative configuration 70a and the positive configuration 70c to provide a combined number of at least three fluoroscopy images of the object 78 from multiple angles. That is, FIG. 6-2 is a first fluoroscopic image captured at the negative configuration 70a with the negative angular offset −θ, FIG. 6-3 is a second fluoroscopic image captured at the neutral configuration 70b with the zero (0°) angle, and FIG. 6-4 is a third fluoroscopic image captured at the positive configuration 70c with the positive +θ angle.

It is noted that while FIGS. 6-2, 6-3, and 6-4 show a bone as a captured object of interest, the object 78 can be any semantically identifiable object. For example, the object 78 may be any part of the endoscope 30 or any anatomical feature including a legion or a nodule. To the extent that the object 78 is composed of denser materials in relation to its surroundings, the object 78 may appear with distinguishable brightness such that a cross-section of the object 78 may be identifiable on a fluoroscopic image, as shown in FIGS. 6-2, 6-3, and 6-4.

In some embodiments, objects appearing in the fluoroscopic images may be segmented to include some identified objects while excluding other objects or features. For instance, the fluoroscopic images of FIGS. 6-2, 6-3, and 6-4 include a bone but excludes other objects and background. In some embodiments, the segmentation can be based on one or more filters, such as a denseness filter or a thickness filter. Regarding filtering, various segmentation algorithms or models may be utilized, including artificial intelligence-driven algorithms/models. In some instances, segmentation may involve decomposition of objects captured in a fluoroscopic image into basis materials such as metal for a scope or tissue for a nodule. Many variations are contemplated.

### Sinogram Generation and Projected Image Simulation

Fluoroscopic sources, such as the source/emitter 75, emit a beam through the object at various angles. Detectors, such as the receiver 74, measure the intensity of the fluoroscopic field after they pass through the object. The data collected at each angle is represented in a sinogram, a two-dimensional array where one axis represents the angle of the beam, and the other represents the position of the detector/receivers 74.

FIGS. 7-1, 7-2, and 7-3 illustrate traditional method of generating a sinogram by varying projection angles in accordance with one or more embodiments. Each of FIGS. 7-1, 7-2, 7-3 illustrate a region of interest 710a-c receiving a corresponding imaging field 716a-c at an angle to generate a respective projected image 718a-c.

The projected image 718a-c can reflect a first object 712a-c and a second object 714a-c, each object having different physical properties that reflect how bright the projected image 718a-c depicts the objects 712, 714. The projected images 718a-c can be appended as slices to another projected image at a neighboring angle to gradually populate the sinogram 720a-c. It is noted that the region of interest 710 is a two-dimensional area and that the projected images 718 is a one-dimensional line. However, the general idea described here in relation to generation of a sinogram can be expanded to a three-dimensional space of interest and a two-dimensional projected image.

Traditionally, the imaging field 716 is applied at a fixed angular interval (e.g., 0.5° interval, 1° interval, 2° interval, etc.) to project images 718 at various angles starting from 0° toward 180° and combine each of the projected images 718 for a complete sinogram.

If a reconstruction of a cross-section of an object is desired, the complete sinogram can be filtered and back-projected to generate the reconstruction. The reconstruction is equivalent of generating a cross-section of the region of interest 710 for a given angle from the completed sinogram 720. One common method for reconstructing cross-sections from sinograms is filtered back projection (FBP). The process involves applying a transform, inverse or otherwise, to the sinogram, applying a filter in the frequency domain to enhance certain features or suppress artifacts, and then back-projecting the filtered data to obtain a two-dimensional image. In addition to FBP, there are other reconstruction algorithms, such as iterative methods. Iterative reconstruction algorithms can iteratively refine an initial estimate of the image to fit the measured sinogram data. Examples include the Maximum Likelihood Expectation Maximization (MLEM) algorithm and the Algebraic Reconstruction Technique (ART).

In some embodiments, it is contemplated that a first cross-section of the first object 712 may be separately reconstructed from a second cross-section of the second object 714 which may also be separately reconstructed for a given angle. Further, the first cross-section and the second cross-section may be merged, overlayed, or otherwise combined to provide a third cross-section depicting both the first object 712 and the second object 714. In contrast with the traditional method, the techniques presented herein can efficiently remove the need to project the object at every angle. That is, unlike the traditional method of generating the sinogram, the techniques may rely on a select few (e.g., three or more) projected images and, from the projected images, simulate a sinogram that is complete for all angles.

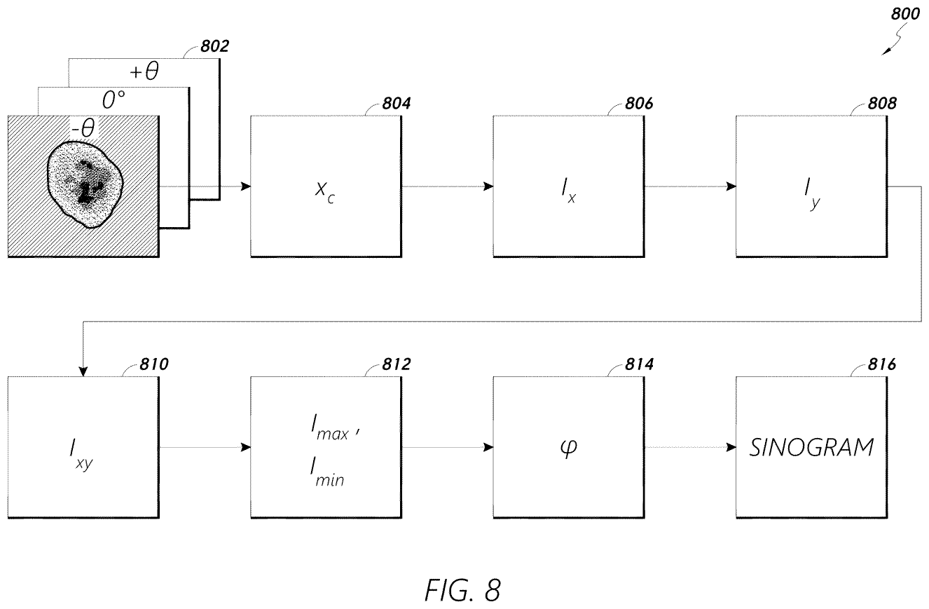

FIG. 8 illustrates a process 800 for simulating a complete sinogram using select few fluoroscopic images in accordance with one or more embodiments. The select few fluoroscopic images may be fluoroscopic images projected at symmetrically rotated known angles, such as the projected images of FIGS. 6-2, 6-3, and 6-4. The process 800 may be implemented at least in part by control circuitry of any of the system components disclosed herein, such as a robotic cart/system and/or control tower/system.

At block 802, the process 800 may involve acquiring or otherwise accessing at least three projected images. In some embodiments, the projected images can be the fluoroscopic images in FIGS. 6-2, 6-3, and 6-4 separated by a fixed angular rotation, for example, by 30° along a rotational axis, symmetrically toward the positive direction and negative direction of the rotational axis. In some embodiments, one object can be identified, segmented, and included in a conditioned image for subsequent use in the process 800 while other objects and features may be subtracted out, as shown in the fluoroscopic images.

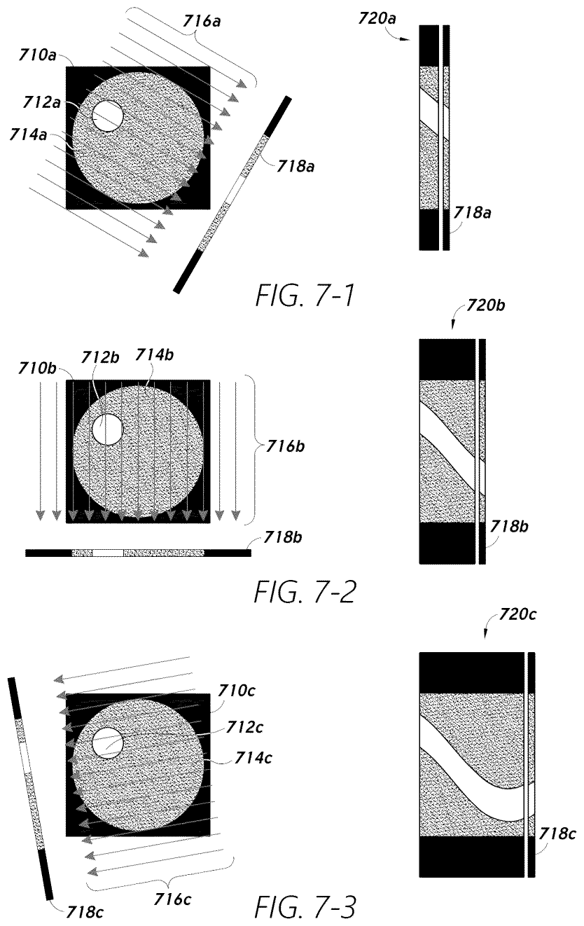

At block 804, centers of masses in the fluoroscopic images may be determined. In each of the fluoroscopic images, the center of mass (x_{c}) in the can be computed based on the following formula:

$${x}_{c} = \frac{Δ p}{{ρ}_{m}} \frac{{\sum }_{i}^{N} {m}_{i} {x}_{i}}{{\sum }_{i}^{N} {m}_{i}} \qquad Equation ( 1 )$$

where Δp is the pixel spacing along the profile (e.g., an axis in the projected image, such as an axis along the linear projected images 718a-c of FIG. 7), mᵢ is the i-th pixel value in the profile, xᵢ is the i-th pixel position, x_{c} is the locus of the center of mass in the orthogonal plane, and ρₘ is a material property known of the projected object. For instance, ρₘ is known for a nodule tissue or a scope material.

At block 806, having computed the center of masses for the projected images, the second moments of area (Iₓ) for planes orthogonal to a given projected image can be determined. In some instances, Iₓ can be computed based on the following formula:

$${I}_{x} = \frac{Δ p}{{ρ}_{m}} {\sum }_{i}^{N} {{m}_{i} ( {x}_{i} - {x}_{c} )}^{2} . \qquad Equation ( 2 )$$

The present disclosure relies on at least three images that are projected from three-dimensional space (3D) onto a two-dimensional plane (2D). A major limitation of using a single 2D projection is that an object cross-sections, such as the scope cross-sections, may not be rotationally symmetric and, hence, moments of inertia (Iₓ) may vary with the angle of projection about the object axis (e.g., scope axis). The operational concept of the mathematical reconstruction is based on the fact that the principal moments of inertia (Iₘₐₓ, Iₘᵢₙ) of an object can be completely determined with only three angularly displaced projections. That is, any two orthogonal projections (Iₓ, I_{y}) can yield a constant defined as the polar moment of inertia (Iₚ) that is equivalent to the sum of the principal moments of inertia (Iₘₐₓ, Iₘᵢₙ). In other words, the polar moment of inertia (Iₚ), which remains constant, is equal to any sum of the two orthogonal moments of inertia (Iₓ, I_{y}) and the sum of the principal moments of inertia (Iₘₐₓ, Iₘᵢₙ). This relationship can be summarized with the following formula:

$${I}_{p} = {I}_{x} + {I}_{y} = {I}_{m ax} + {I}_{m i n} . \qquad Equation ( 3 )$$

At block 808, the process 800 may involve determining the moment of inertia orthogonal to Iₓ, the orthogonal moment of inertia referenced as I_{y}. As described in relation to FIG. 6, in an environment with a C-arm structure, it is generally possible to acquire two additional projected images at the same rotational angle ±θ on either side (e.g., symmetrically positioned) of the original projected image about the C-arm axis. The present disclosure utilizes the two additional projected images in connection with the rotational axis theorem to determine I_{y}. Specifically, the rotational axis theorem provides that the moment of inertia at angle θ can be given by:

$${I}_{θ} = \frac{{I}_{x} + {I}_{y}}{2} + \frac{{I}_{x} - {I}_{y}}{2} cos ( 2 θ ) - {I}_{xy} sin ( 2 θ ) \qquad Equation ( 4 )$$

where I_{xy} is the product of inertia, and I_{y} is the moment of inertia orthogonal to Iₓ. Computing I_{θ+} and I_{θ-} individually using the Equation (4) for θ+ and θ−, summing I_{θ+} and I_{θz}, and finally solving for I_{y} provides:

$${I}_{y} = \frac{{I}_{θ +} + {I}_{θ -} - 2 {I}_{x} {cos}^{2} θ}{2 {sin}^{2} θ} \qquad Equation ( 5 )$$

Accordingly, I_{y} can be determined based on the two additional projected images, the known Iₓ from Equation (2), and Equation (4).

At block 810, the process 800 may involve determining the product of inertia (I_{xy}). Using either of I_{θ+} or I_{θ-} to solve for I_{xy} with Equation (4) provides:

$${I}_{xy} = \frac{\frac{{I}_{x} + {I}_{y}}{2} + \frac{{I}_{x} - {I}_{y}}{2} cos ( 2 θ ) - {I}_{θ}}{sin ( 2 θ )} . \qquad Equation ( 6 )$$

It is noted that the two orthogonal projections (Iₓ, I_{y}) are known. Angle (θ) and the moment of inertia (I_{θ}) at the angle (θ) is known. Thus, I_{xy} can be determined by substituting the known values into Equation (6).

At block 812, the process 800 may involve determining the principal moments of inertia (Iₘₐₓ, Iₘᵢₙ). In three-dimensional space, for any arbitrary shape assuming uniform mass distribution, there are always three perpendicular axes through any point, usually the center of mass for convenience, for which the moments of inertia are maximized or minimized. These axes are known as the principal axes. The moments of inertia about these principal axes are referred to as the principal moments of inertia which are, here, Iₘₐₓ and Iₘᵢₙ. The principal moments of inertia, Iₘₐₓ and Iₘᵢₙ, can be determined based on the previously determined moments of inertia (Iₓ, I_{y}, and I_{θ}) as follows:

$${I}_{m ax} = \frac{{I}_{x} + {I}_{y}}{2} + \sqrt{{( \frac{{I}_{x} - {I}_{y}}{2} )}^{2} + {I}_{xy}^{2}} , \qquad Equation ( 7 )$$

$${I}_{m i n} = \frac{{I}_{x} + {I}_{y}}{2} \sqrt{{( \frac{{I}_{x} - {I}_{y}}{2} )}^{2} + {I}_{xy}^{2}} . \qquad Equation ( 8 )$$

At block 814, the process 800 may involve determining the principal angle (φ) of Iₘₐₓ to the reference coordinate system. When it is assumed that Iₓ and I_{y} are about the reference coordinate system, φ can be computed by:

$$φ = - \frac{1}{2} a tan ( \frac{2 {I}_{xy}}{{I}_{x} - {I}_{y}} ) . \qquad Equation ( 9 )$$

At block 816, the process 800 may involve generating a sinogram. In some embodiments, the principal moments of inertia (Iₘₐₓ, Iₘₐₓ) and the product of inertia (I_{xy}) can be used to generate the sinogram. Specifically, Iₘₐₓ, Iₘₐₓ, and I_{xy} can be used to compute second moments of area for all angles that were not measured (e.g., not involved with the projected images) until a complete ensemble of projections can be produced at a fixed interval (e.g., a 1° interval) for projections from 0° toward 180°. The complete ensemble can be the sinogram of a particular object (e.g., only a scope, only a nodule, etc.) for all projection angles φ from 0° toward 180°.

Complete data will consist of an object thickness distribution, such as the scope thickness distribution having uniform density, at each angle. That is, the thickness distribution may be generated based on an assumption that the object has a uniform density profile. The object thickness distribution may be characterized by total area (A, generally fixed for all projections of the same imaging device), center of mass position (c_{φ}), and the second moment of area at angle φ (I_{φ}). From the math above, everything is known about each projection except the actual distribution. Subsequent procedures will begin with generating or simulating a distribution for all of the missing projections corresponding to the known A, Iφ, and c_{φ}.

The generated scope thickness profiles are first fitted to a function with a sufficient number of vertices so that fit metric may provide a substantial fit, such as the least squares fit of at least R²≥99% or other sufficient fit value, while retaining the original A, I_{φ}, and c_{φ} to an acceptable error. The function can include a polynomial function, a spline function, or the like. It is contemplated that non-uniform rational basis spline (NURBS) is a model using basis splines (B-splines) that can be utilized as a fitting function for the purpose, although various other functions are contemplated.

The generation of the distribution can begin from the projected image at the neutral configuration, which is the image with known (e.g., measured) properties at angle φ=0°. Beginning with the fitted distribution of φ=0°, vertices of the fitting function can be iteratively adjusted for the simulated projected image at angle φ=1° while retaining the original A and c_{φ} until Iφ satisfies a condition associated with a threshold value, such as rising above, falling below, or reaching the threshold value. Projected images at φ=2° can be simulated with the similar iterative adjustment beginning with the fitted distribution of φ=1°, and so forth. Similar process can be repeated for each missing projected images at other angles and also, toward negative angles, until the sinogram is complete.

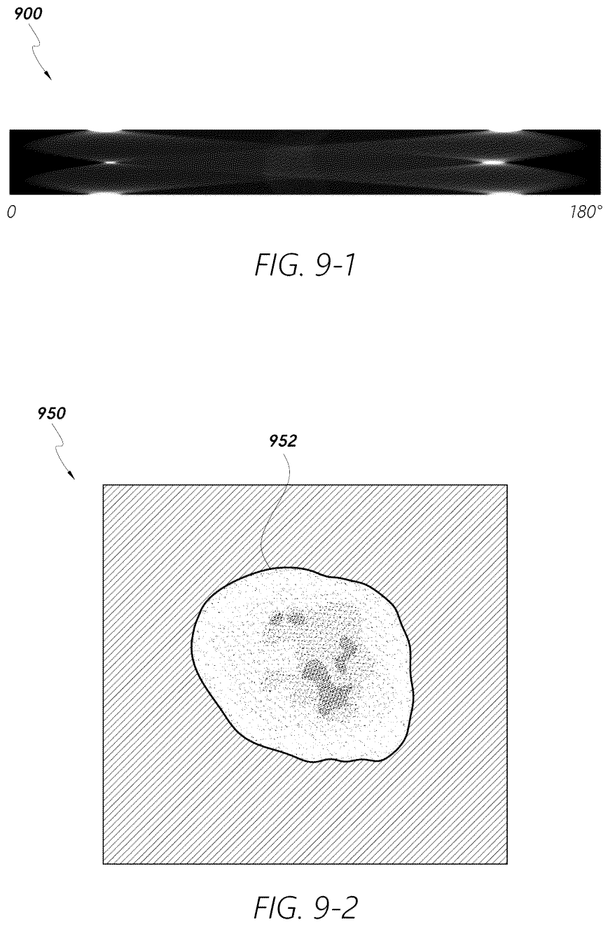

Thus, in contrast with the traditional method, the techniques of the present disclosure can utilize Equations (1)-(9) with three projected images to simulate other projected images and complete a sinogram. For example, FIG. 9-1 illustrates a completed sinogram 900 generated from the process 800 applied the three to fluoroscopic images of a bone of FIGS. 6-2, 6-3, and 6-4.

### Sinogram and Reconstructed Cross-Sections of Objects

FIGS. 9-1 and 9-2 illustrate an example simulated sinogram 900 and a reconstructed cross-section 950 of an object from the sinogram in accordance with one or more embodiments. FIG. 9-1 shows a complete sinogram of an object generated with the process 800 of FIG. 8. As mentioned, sinograms are representations of fluoroscopic attenuation measurements taken at various different angles around an object. FIG. 9-2 shows a reconstructed cross-section of an object 952. Generally, reconstruction of an object involves a sinogram from which a cross-sectional slice of an object may be acquired. Reconstruction in the context of fluoroscopic sinograms refers to the process of converting the collected raw data, in the form of sinograms, into a meaningful image, such as a cross-section of the object at a given angle.

It is noted that the example sinogram 900 and the reconstructed cross-section 950 of the object 952 are of a bone but, as described previously, the object 952 can be any semantically segmentable object including without limitation, a scope, a scope tip or any portion of the scope, a nodule tissue, or any other anatomical features. The reconstruction 950 showing the object 952 at a zero angle confirms that the reconstruction 950 closely matches the actual projected image of the object 952 at zero angle (e.g., a neutral configuration 70b of FIG. 6-3).

In some embodiments, the reconstruction 950 can involve filtering and back-projection performed on the simulated sinogram 900. For example, FIG. 9-2 illustrates the reconstruction 950 of a cross-section of the bone from the sinogram 900. Similar filtering and back-projection can be applied to reconstruct other objects from the same or another similarly generated sinogram. Accordingly, a reconstructed cross-section may be repeated with various objects until the entire cross-section is reconstructed. Specifically, it is contemplated that a scope (including a tip) and a nodule may be reconstructed from three projected images.

The reconstructed cross-sectional images can include a mathematical reconstruction of the basis objects/materials (e.g., a scope vs. a nodule) in the images and the images can be used to derive various geometrical properties of the objects and materials within the image(s). The derived properties, in turn, can serve to initialize object semantic segmentation and indicate to a user viewing the projected and simulated images a more accurate identification of the objects. For example, derived geometrical properties including principal moments of inertia, second moments of area, an object's center of mass (which may be close to a centroid of the object in instances where the object is homogeneous), axis of rotation, or other properties may be displayed to a user in association with the image and help identify objects and their properties. As another example, some derived properties such as a center of mass of a scope tip can help estimate pose of the scope tip in the image.

### Object Semantic Segmentation Using the Sinogram

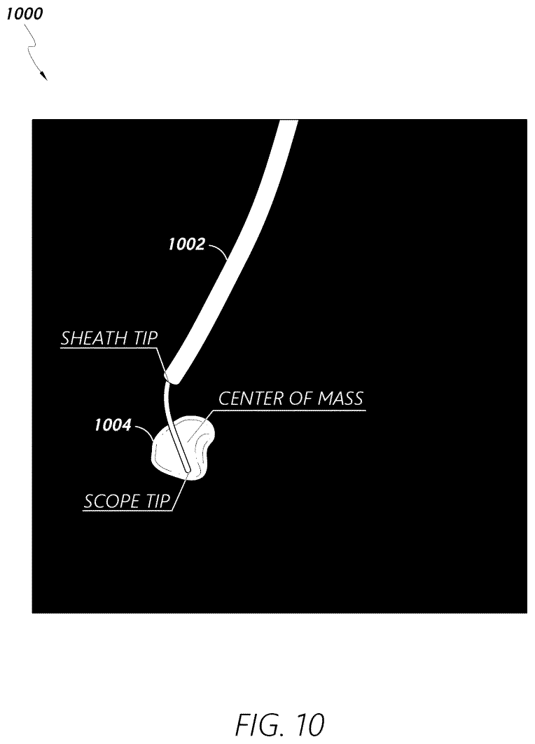

FIG. 10 illustrates a presentation 1000 of derived geometrical properties of a fluoroscopic image in accordance with one or more embodiments. In some embodiments, the presentation 1000 can be provided on the one or more display(s) 56 for review by the physician 5. In some other embodiments, the presentation 1000 can include a reconstructed cross-section (e.g., a simulated projection image) that is simulated in a manner consistent with the present disclosure. In some other embodiments, the presentation 1000 can include an actual projected image associated with a known angle.

The presentation 1000 can include at least one reconstructed cross-section. The reconstructed cross-section can be displayed on a computer monitor or printed for analysis. In some implementations, modern medical imaging systems may provide detailed three-dimensional reconstructions as well.

The reconstructed object can be a scope 1002 or any portions thereof (e.g., a scope tip, a sheath tip, a biopsy needle, basket, forceps, etc.) as well as various anatomical feature(s) 1004 (e.g., a nodule, a lesion, a tissue, etc.). Additionally, the presentation 1000 can include an indicator referencing the reconstructed object and present the indicator in association with the object. For example, the indicator can be a location indicator referencing the center of mass or locations of other physical properties regarding the object. As another example, the indicator can reference one or more principal axes and provide a navigation software with valuable information regarding orientation of the object, such as the scope orientation. In some embodiments, the accurate reconstruction of the objects can help segmentation and detection of one or more objects.

### Reconstruction and Position Estimation Initialization

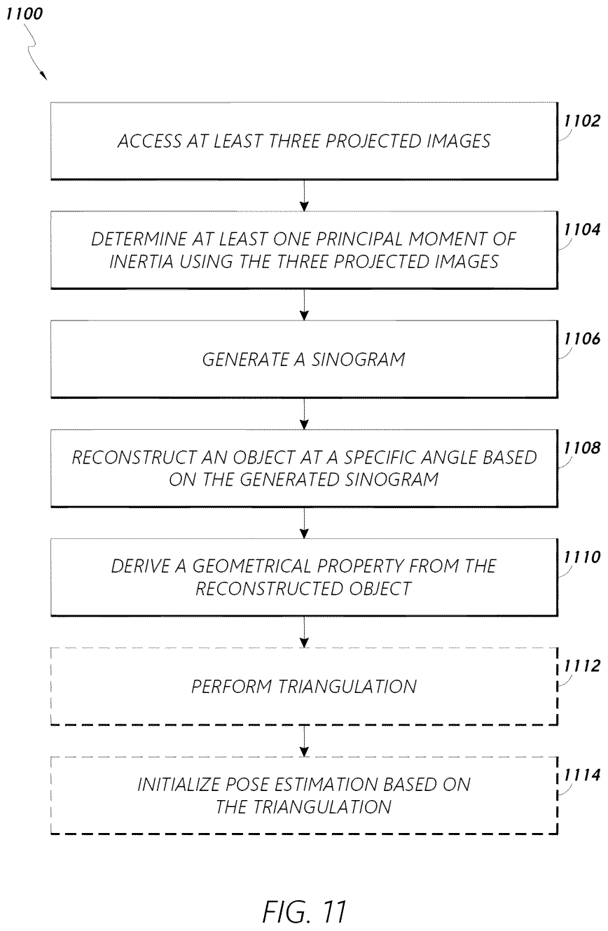

FIG. 11 illustrates a flowchart of a process 1100 of reconstructing an object from fluoroscopic images in accordance with one or more embodiments. For example, the process 1100 may be implemented in connection with reconstruction of an object, such as a scope tip and a nodule, and initialization of later pose estimation of the objects. The process 1100 may be implemented at least in part by control circuitry of any of the system components disclosed herein, such as a robotic cart/system and/or control tower/system.

At block 1102, the process 1100 may involve accessing at least three projected images. As described, the projected images can be axially rotated images generated by a fluoroscopic imaging device attached to a C-arm at known, symmetric, angular offset. For instance, the projected images can be the cross-sections shown in FIGS. 6-2, 6-3, 6-4.

At block 1104, the process 1100 may involve determining at least one principal moment of inertia using the three projected images. In some implementations, an object of interest may be left included while other objects or features may be excluded from the three projected images. Specifically, the principal moments of inertia (Iₘₐₓ, Iₘᵢₙ) can be determined from the three projected images based on the rotational axis theorem.

At block 1106, the process 1100 may involve generating a sinogram. Here, for remaining angles other than the three angles associated with the three projected images, projected images of the remaining angles can be simulated based on the at least one principal moment of inertia. In some implementations, the projected images can be simulated at fixed intervals for 0° toward 180° to complete the sinogram such that the completed sinogram may consist of a thickness distribution of an object at each angle. In some implementations, simulating the projected images for each adjacent angle may involve iteratively adjusting a number of vertices of a function fitted to a projected image of the object projected at a previous angle. The projected image at the previous angle may be based on an original projected image or a simulated projected image.

At block 1108, the process 1100 may involve reconstructing an object as seen from a specific angle based on the generated sinogram. In some implementations, the reconstruction may involve some post-processing steps. As an example of a post-processing step, the reconstruction may involve an inverse transformation. The actual reconstruction process can be quite complex based on various post-processing algorithms and steps employed, the specific imaging technique, and equipment used. The choice of reconstruction method can affect image quality, spatial resolution, and the ability to distinguish different structures within the imaged object.

At block 1110, the process 1100 may involve deriving a geometrical property from the reconstructed object. Some example derivable geometrical property includes a centroid of the object, moments of inertia, second moment of area, or the like. In some implementations, the object can be annotated in the original projected image or the simulated projected image based on the derived geometrical property.

Optionally, the process 1100 may involve determining a three-dimensional position of the object, for example, the distal tip of a scope or a nodule centroid. The three-dimensional position of the object may later be used as an initialization for pose estimation algorithms. When determining such three-dimensional position of the object, the process 1100 may involve performance of some additional blocks.

At block 1112, the process 1100 may involve performing triangulation. Triangulation is a common method of projecting points from a two-dimensional plane to a three-dimensional space using, in some implementations, point-of-capture parameters. In fluoroscopic applications, the point-of-capture parameters may be determined based on a C-arm position or the fluoroscopic imaging device attached to the C-arm. Accordingly, such parameters determined during installation and/or calibration may be used to perform triangulation.

At block 1114, the process 1100 may involve initializing pose estimation based on the triangulation. For example, a three-dimensional position of the object may be determined based on the triangulation and can serve as an initial estimated pose of the object. The initial estimated pose may be presented to a user on a display in association with a projected image.

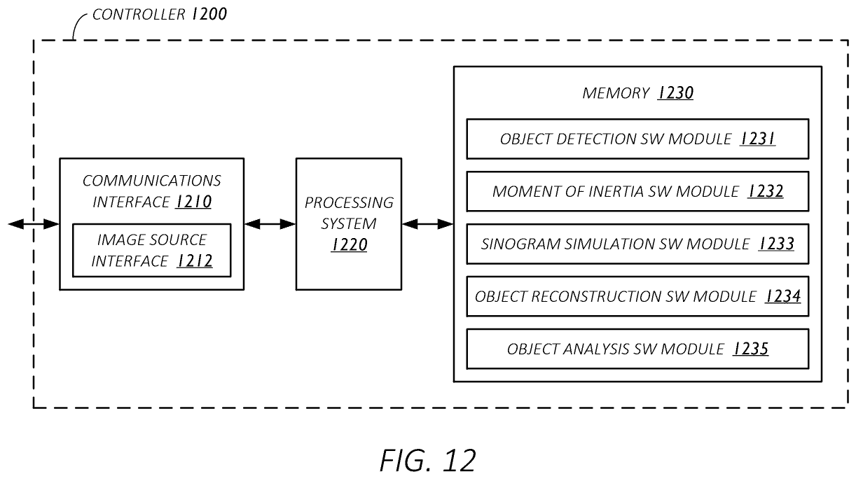

FIG. 12 shows a block diagram of an example controller 1200 for a medical system, according to some implementations. In some implementations, the controller 1200 may be one example of any of the control circuitry 211 and/or control circuitry 251 of FIG. 4.

The controller 1200 includes a communication interface 1210, a processing system 1220, and a memory 1230. The communication interface 1210 is configured to communicate with one or more components of the medical system. For example, the communication interface 1210 may include an image source interface 1212 for communicating with one or more image sources (such as the fluoroscopy imaging system 70 of FIGS. 1-3 and 5). In some implementations, the image source interface 1212 may receive a plurality of images associated with a sinogram, where the plurality of images is captured by an imaging device at a plurality of angles, respectively, along a rotational axis of the imaging device.

The memory 1230 includes a non-transitory computer-readable medium (including one or more nonvolatile memory elements, such as EPROM, EEPROM, Flash memory, or a hard drive, among other examples) that stores the following software (SW) modules: an object detection SW module 1231 to detect an object in each image of the plurality of images; a moment of inertia SW module 1232 to determine a moment of inertia associated with the object based on the plurality of images; a sinogram simulation SW module 1233 to simulate one or more images for the sinogram based at least in part on the moment of inertia associated with the object; an object reconstruction SW module 1234 to generate a reconstruction of the object based on the sinogram; and an object analysis SW module 1235 to determine one or more properties of the object based on the reconstruction. Each of the software modules 1231-1235 includes instructions that, when executed by the processing system 1220, causes the controller 1200 to perform the corresponding functions.

The processing system 1220 may include any suitable one or more processors capable of executing scripts or instructions of one or more software programs stored in the controller 1200 (such as in the memory 1230). For example, the processing system 1220 may execute the object detection SW module 1231 to detect an object in each image of the plurality of images. The processing system 1220 also may execute the moment of inertia SW module 1232 to determine a moment of inertia associated with the object based on the plurality of images. The processing system 1220 may execute the sinogram simulation SW module 1233 to simulate one or more images for the sinogram based at least in part on the moment of inertia associated with the object. The processing system 1220 may further execute the object reconstruction SW module 1234 to generate a reconstruction of the object based on the sinogram. Still further, the processing system 1220 may execute the object analysis SW module 1235 to determine one or more properties of the object based on the reconstruction.

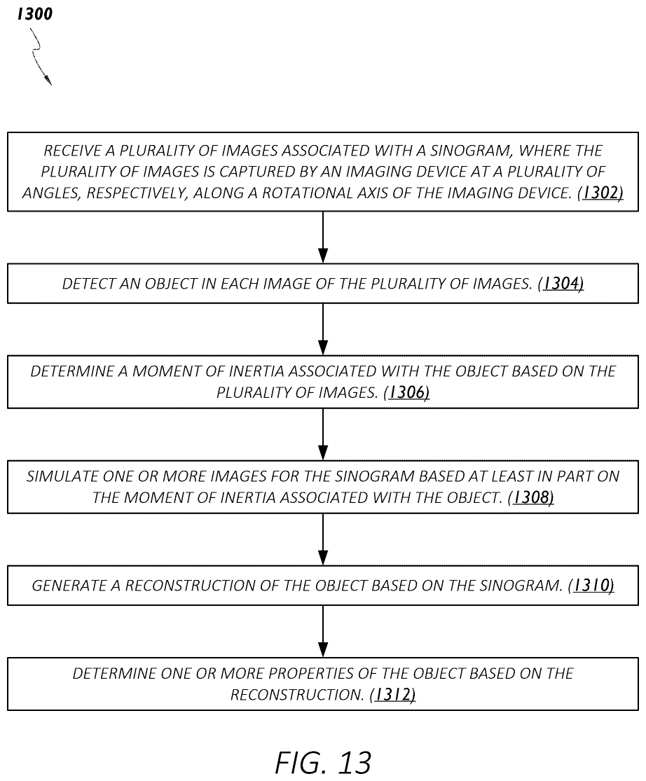

FIG. 13 shows an illustrative flowchart depicting an example operation 1300 for determining one or more properties of an object, according to some implementations. In some implementations, the example operation 1300 may be performed by a controller for a medical system such as the controller 1200 of FIG. 12 or any of the control circuitry 211 and/or 251 of FIG. 4.

The controller receives a plurality of images associated with a sinogram, where the plurality of images is captured by an imaging device at a plurality of angles, respectively, along a rotational axis of the imaging device (1302). In some implementations, the imaging device may include a fluoroscopy system. The controller detects an object in each image of the plurality of images (1304). In some implementations, the object may include an instrument, configured to be driven through a luminal network, or an anatomical feature. The controller further determines a moment of inertia associated with the object based on the plurality of images (1306).

The controller simulates one or more images for the sinogram based at least in part on the moment of inertia associated with the object (1308). The controller generates a reconstruction of the object based on the sinogram (1310). In some implementations, the reconstruction may include a cross-section of the object. The controller further determines one or more properties of the object based on the reconstruction (1312). In some implementations, the controller may further present a location indicator referencing the object on a display.

In some implementations, the plurality of images may include a first image captured at a first angle of the plurality of angles, a second image captured at a second angle of the plurality of angles, and a third image captured at a third angle of the plurality of angles, where the first angle and the second angle are separated by the same degree of angular rotation as between the second angle and the third angle. In some implementations, adjacent angles of the plurality of angles may be separated by a fixed interval.

In some aspects, the sinogram may include a thickness distribution for the object over the plurality of angles. In some implementations, the controller may further determine a fitting function, having a plurality of vertices, that fits a known thickness profile to the thickness distribution for the object. In some implementations, the simulating of each image of the one or more images for the sinogram may include iteratively adjusting the plurality of vertices of the fitting function until a second moment of area at the angle associated with the simulated image satisfies a threshold condition.

In some implementations, the controller may further perform a semantic segmentation operation based at least in part on the one or more properties of the object determined based on the reconstruction. In some implementations, the controller may further determine an axis associated with the moment of inertia, determine a second moment of area associated with an axis orthogonal to the axis associated with the moment of inertia, and determine a principal angle associated with the object based on the moment of inertia and the second moment of area.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A system comprising:
an imaging device configured to rotate axially about a rotational axis and to capture a plurality of images associated with a sinogram at a plurality of angles, respectively, along the rotational axis;
one or more processors; and
a memory storing instructions that, when executed by the one or more processors, cause the system to:
detect an object in each image of the plurality of images;
determine a moment of inertia associated with the object based on the plurality of images;
simulate one or more images for the sinogram based at least in part on the moment of inertia associated with the object;
generate a reconstruction of the object based on the sinogram; and
determine one or more properties of the object based on the reconstruction.

2. The system of claim 1, wherein execution of the instructions further causes the system to:
present a location indicator referencing the object on a display.

3. The system of claim 1, wherein the object comprises an instrument, configured to be driven through a luminal network, or an anatomical feature.

4. The system of claim 1, wherein the imaging device comprises a fluoroscopy system.

5. The system of claim 1, wherein the plurality of images include a first image captured at a first angle of the plurality of angles, a second image captured at a second angle of the plurality of angles, and a third image captured at a third angle of the plurality of angles, the first angle and the second angle being separated by the same degree of angular rotation as between the second angle and the third angle.

6. The system of claim 1, wherein adjacent angles of the plurality of angles are separated by a fixed interval.

7. The system of claim 1, wherein the sinogram comprises a thickness distribution for the object over the plurality of angles.

8. The system of claim 7, wherein execution of the instructions further causes the system to:
determine a fitting function, having a plurality of vertices, that fits a known thickness profile to the thickness distribution for the object.

9. The system of claim 8, wherein the simulating of each image of the one or more images for the sinogram comprises:
iteratively adjusting the plurality of vertices of the fitting function until a second moment of area at the angle associated with the simulated image satisfies a threshold condition.

10. The system of claim 1, wherein the reconstruction comprises a cross-section of the object.

11. The system of claim 1, wherein execution of the instructions further causes the system to:
perform a semantic segmentation operation based at least in part on the one or more properties of the object determined based on the reconstruction.

12. The system of claim 1, wherein execution of the instructions further causes the system to:
determine an axis associated with the moment of inertia;
determine a second moment of area associated with an axis orthogonal to the axis associated with the moment of inertia; and
determine a principal angle associated with the object based on the moment of inertia and the second moment of area.

13. A computer-implemented method comprising:
receiving a plurality of images associated with a sinogram, the plurality of images being captured by an imaging device at a plurality of angles, respectively, along a rotational axis of the imaging device;
detecting an object in each image of the plurality of images;
determining a moment of inertia associated with the object based on the plurality of images;
simulating one or more images for the sinogram based at least in part on the moment of inertia associated with the object;
generating a reconstruction of the object based on the sinogram; and
determining one or more properties of the object based on the reconstruction.

14. The method of claim 13, further comprising presenting a location indicator referencing the object on a display.

15. The method of claim 13, wherein the plurality of images include a first image captured at a first angle of the plurality of angles, a second image captured at a second angle of the plurality of angles, and a third image captured at a third angle of the plurality of angles, the first angle and the second angle being separated by the same degree of angular rotation as between the second angle and the third angle.

16. The method of claim 13, wherein adjacent angles of the plurality of angles are separated by a fixed interval.

17. The method of claim 13, wherein the sinogram comprises a thickness distribution for the object over the plurality of angles, the method further comprising determining a fitting function, having a plurality of vertices, that fits a known thickness profile to the thickness distribution for the object.

18. The method of claim 17, wherein the simulating of each image of the one or more images for the sinogram comprises iteratively adjusting the plurality of vertices of the fitting function until a second moment of area at the angle associated with the simulated image satisfies a threshold condition.

19. The method of claim 13, wherein the object comprises at least one of a medical instrument or a nodule.

20. A controller for a medical system comprising:
one or more processors; and
a memory storing instructions that, when executed by the one or more processors, cause the controller to:
receive a plurality of images associated with a sinogram, the plurality of images being captured by an imaging device at a plurality of angles, respectively, along a rotational axis of the imaging device;
detect an object in each image of the plurality of images;
determine a moment of inertia associated with the object based on the plurality of images;
simulate one or more images for the sinogram based at least in part on the moment of inertia associated with the object;
generate a reconstruction of the object based on the sinogram; and
determine one or more properties of the object based on the reconstruction.
